(19) United States
(12) Patent Application Publication
(10) Pub. No.: US 2009/0075289 A1
(43) Pub. Date: Mar. 19, 2009

(54) Collection Assembly for Obtaining Oral Samples From an Animal

(76) Inventors: Yao-Hua Zhang, Los Angeles, CA (US); Bing-Ling Huang, Los Angeles, CA (US); Edward R.B. McCabe, Pacific Palisades, CA (US); Nicole MaClennan, Malibu, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/293,274

(22) PCT Filed: Mar. 16, 2007
(86) PCT No.: PCT/US2007/064240

§ 371 Date: Sep. 16, 2008

Related U.S. Application Data
(60) Provisional application No. 60/783,456, filed on Mar. 17, 2006.

(51) Int. Cl. A61B 5/00; C12Q 1/68

(52) U.S. Cl. 435/6; 600/582

(57) ABSTRACT

The present invention provides a collection assembly for obtaining a sample from within the mouth of an animal. The collection assembly comprises a collection wand, having a scoop-shaped collecting end, and, optionally, an oral speculum. Also provided are methods of using the collection assembly to obtain an oral sample from the mouth of an animal without causing significant discomfort to the animal.

### RELATED APPLICATION

This application claims priority from Unites States Provisional Application Ser. No. 60/783,456 filed on Mar. 17, 2006, the entire contents of which are hereby incorporated by reference.

### FIELD OF THE INVENTION

The present invention pertains to the field of sample collection devices. More particularly, the present invention pertains to the field of oral sample collection devices for use in animals.

### BACKGROUND

Obtaining cells from newborn rodents is critical for investigations involving transgenic and knockout mice. Various practices exist for obtaining cells from animals for DNA genotyping. For example, common practices used to acquire cells from a neonatal mouse for DNA genotyping involve total or partial amputation of the rodent's toe, ear or tail. These practices are both invasive and mutilating, and toe clipping has been generally banned. Furthermore, in response to these practices, mice exhibit responses to pain that may include one or more specific behaviors such as vocalizing, biting and avoidance (running away). Neonatal mice contort and move their entire bodies back and forth in response to tail clipping, showing that they experience significant pain.

Non-invasive and painless buccal cell isolation methods involving saliva, mouthwash, treated filter paper, cytobrush and foam or cotton-tip swabs are available for collecting buccal cells from humans for diagnostic analyses (1,6,7). These methods, however, are extremely difficult, if not impossible, to adapt for the small size of, for example, the newborn mouse. For example, the filter paper on the Bode Buccal DNA collector is about the width of a neonatal mouse head (1). The Oragene™ saliva collection device requires 1 ml of saliva sample to extract sufficient DNA for processing, or approximately the total weight of the 1 g neonatal mouse (DNA Genotek Inc.). Traditional cotton swabs are much too large to collect buccal cells from a day of life (DOL) 1 mouse and would have the potential to suffocate the mouse if forced into the mouth. Although comparison of cytobrush, mouthwash and treated card for obtaining human buccal cells found that the cytobrush was the best method for human sampling (6), these approaches cannot be adapted directly to mice, especially DOL1 mice. A serrated pipette tip used to collect human cells for RNA isolation is too abrasive for the fragile DOL1 mouse cheek (7). Mouse pups require methods of handling that minimize pain and stress, since they are very fragile and can die easily during execution of the experimental protocol.

Protocols have been developed to address minimally invasive and painless mouse buccal cell sampling. One method for sampling and extraction of mouse DNA used a cotton swab adapted for adult, but not neonatal, mice (4). A common toothpick, though more appropriate in size, would lack a reservoir for collecting adequate buccal cells from the newborn mouse for subsequent analyses. Kits involving a buccal brush or swab (Epicentre), mouse saliva (10 μl; Sigma), and buccal cells applied on a card with a swab (Whatman) are examples of commercial products available for adult mice. However, the youngest mouse described in the protocols for these products was 1 month old.

To date, known, standardized, relatively non-invasive methods for buccal cell sampling from newborn mice are not available from commercial sources or in the published literature. Therefore, there is a need for a less invasive, non-mutilating approach to obtain cells from animals, such as neonatal mice, for experimental procedures that require an oral sample, such as buccal cell sampling, for example for DNA extraction and genotyping.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

In accordance with one aspect of the present invention, there is provided a sample collection assembly, comprising a collection wand having an operation end and a collection end, said collection end having a scoop-shaped portion configured for collecting an oral sample from within the mouth of an animal wherein said scoop-shaped portion is sized and shaped to fit within the mouth of said animal without causing significant discomfort to said animal. The sample collection assembly optionally includes an oral speculum.

In accordance with another aspect of the present invention, there is provided an oral speculum, comprising a resilient handle portion having a first end comprising a first speculum blade and a second end comprising a second speculum blade, said resilient handle urges said first speculum blade and said second speculum blade from a closed position to an open position, said first speculum blade and said second speculum blade are sized to fit within the mouth of an animal, wherein in said open position the distance between said first speculum blade and said second speculum blade is sufficient to maintain the mouth of said animal open.

In accordance with another aspect of the present invention, there is provided a method of collecting an oral sample from an animal, comprising (a) gently maintaining the mouth of the animal open (b) collecting the oral sample from the mouth of the animal using a collection assembly according to the present invention.

In accordance with another aspect of the present invention, there is provided a kit comprising (a) a collection assembly according to the present invention; and (b) instructions for the use thereof.

In accordance with another aspect of the present invention, there is provided a kit comprising (a) a collection assembly according to the present invention, (b) an oral speculum according to the present invention and (c) instructions for the use thereof.

### BRIEF DESCRIPTION OF THE FIGURES

FIG. 1 depicts an example of (Panel A) an oral speculum of the present invention, and (Panel B) use of this oral speculum to open the mouth of a neonatal mouse;

FIG. 2 depicts an example of (Panel A) a sample collection wand of the present invention, and depicts an example of a sample collection assembly of the present invention including a collection wand having a lid, and a sample collection tube in a partially open condition (Panel B) and in a closed condition (Panel C);

FIG. 3 includes photographs showing (Panel A) insertion of an oral speculum according to one embodiment of the present invention into the mouth of a neonatal mouse, and (Panel B) sample collection from the mouth of a neonatal mouse using a sample collection assembly including a speculum and a collection wand according to one embodiment of the present invention;

FIG. 4 is a photograph of buccal cells obtained from a day of life 1 (DOL1) neonatal mouse using a collection assembly according to one embodiment of the present invention;

FIG. 5 is a photograph of a gel showing (Panel A) sex-typing PCR of DOL1 neonatal mice: 1 kb ladder (lane 1), Male (lane 2) and female (lane 3), (Panel B) dilutions of male mouse DNA showing sensitivity to <11 of a 1:10 dilution of the neutralized lysis extract, showing ample DNA for >50 PCR sex-typing amplifications was obtained using a method and a sample collection assembly according to one embodiment of the present invention; and

FIG. 6 is a photograph showing genotyping PCR of Gyk knockout, carrier and wild type mice: using DNA from samples obtained using a method and collection assembly according to one embodiment of the present invention Panel A, shows genotyping PCR of Gyk wild type and carrier mice and Panel B shows genotyping PCR of knockout and wild type mice.

### DETAILED DESCRIPTION OF THE INVENTION

As will be described in more detail below, the present invention is directed to a sample collection assembly comprising a collection wand sized and configured for sample collection from within the mouth of an animal without causing significant discomfort to the animal. The sample collection assembly optionally includes an oral speculum and/or a collection tube. The present invention further pertains to a method of use of the collection assembly to obtain an oral sample from an animal.

Discomfort in an animal, in particular a laboratory animal, is observed by, for example, struggle and/or movements back and forth and/or vocalizations and avoidance by the animal, which is typically observed when the animal is in pain or under stress. Such discomfort is observed in known mutilating procedures such as total or partial amputation of the laboratory animal's toe, ear or tail. Similar or corresponding discomfort reactions in response to pain or stress are observed in both neonatal and non-neonatal animals, and would be readily recognizable by the skilled worker. The methods and devices of the present application permit an oral sample to be obtained from the animal without causing discomfort or without causing significant discomfort. A worker skilled in the art, having experience working with animals, would readily appreciate what is meant by “significant discomfort”. In an animal experiencing no discomfort there would be no observation of the discomfort reactions described above. In an animal experiencing mild discomfort, but not significant discomfort, some discomfort reactions would be observed but the discomfort should be tolerable to the animal.

The term “oral sample”, as used herein, includes samples obtained from within the mouth of an animal that contains one or more of: saliva, sputum, microorganisms (including viral, bacterial, fungal, protozoan, parasitic, single-celled, and/or multi-cellular organisms) present in the mouth, cells, buccal mucosa and/or buccal cells.

The term “sputum”, as used herein, refers to mucoid matter contained in or discharged from the nasal or buccal cavity of a mammal, including saliva and discharges from the respiratory passages, including the lungs.

The term “saliva”, as used herein, refers to the secretion, or combination of secretions, from any of the salivary glands, including the parotid, submaxillary, and sublingual glands, optionally mixed with the secretions from the numerous small labial, buccal, and palatal glands that line the mouth.

For descriptive purposes herein, one embodiment of a sample collection assembly of the invention will be described with reference to the figures. The illustrated embodiments may be particularly suited for collecting an oral sample from the mouth of a neonatal mouse, but would be readily adaptable for use in other animals, including neonatal and non-neonatal laboratory animals, such as mice, rats, hamsters and rabbits. Thus, according to this embodiment, the collection tip at the working end can be passed into the mouth of a mouse or other species and directed towards a part of the mouth such as the inner cheek or buccal pouch. Once at a desired location the collection wand can be manipulated to facilitate sample collection.

In the drawings, like reference numerals represent like parts and assemblies throughout the several views. Reference to the drawings is not intended to limit the scope of the invention.

### Collection Assembly

### Collection Wand

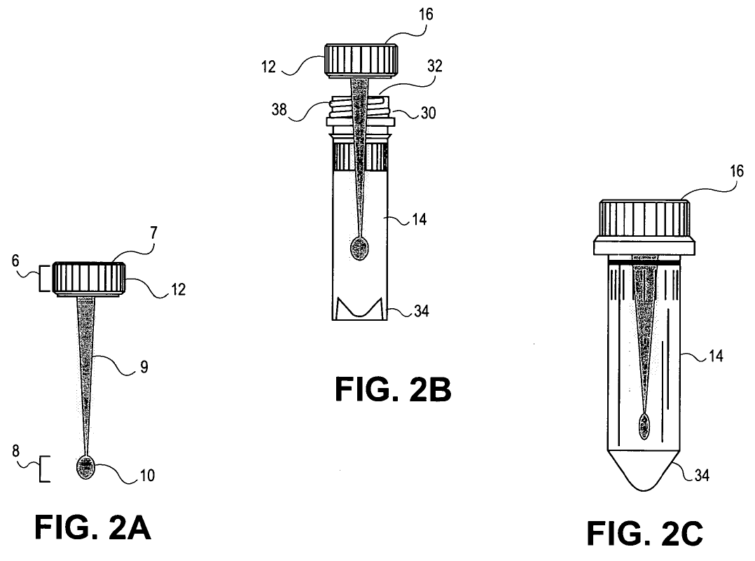

Referring to FIGS. 2A-2C, the collection assembly comprises collection wand 4 having operation end 6 and collection end 8. Operation end 6 can include a handle 12 configured to fit and/or orient with the operator's hand. The shape and length of handle 12 will vary according to the intended use, needs, preferences and/or choice of construction materials.

A shaft 9 between operation end 6 and collection end 8 can be of any length sufficient to reach the sample source and can be optimized to facilitate ease of use depending on operator requirements and/or the animal from which the sample is being collected. In a particular example, shaft 9 is sized such that the length of collection wand 4 is about 3 cm in length.

Collection end 8 includes scoop-shaped portion 10 configured for collecting an oral sample from within the mouth of an animal. Scoop-shaped portion 10 is sized and shaped to fit within the mouth of said animal without causing significant discomfort to said animal. The dimensions of scoop-shaped portion 10 are determined based on the average size of the mouth of the animal (species and age) from which the sample is to be collected. For example, in the case of a neonatal mouse, an appropriate size is determined by measuring the distance between the gum line of the upper and lower jaw at each jaw's mid-point. A skilled worker would readily be able to obtain similar measurements in other animals. In the case of a neonatal mouse, scoop-shaped portion 10 is less than about 3 mm in width. In one example, in the case of a DOL1 neonatal mouse, scoop-shaped portion 10 is less than about 2 mm in width. In another example, in the case of a DOL2 and DOL3 neonatal mouse, scoop-shaped portion 10 is less than about 3 mm in width. In the case of a DOL7 neonatal mouse, scoop-shaped portion 10 is less than about 5 mm in width. In the case of a DOL14 neonatal mouse, scoop-shaped portion 10 is less than about 5 mm in width. In the case of a DOL21 neonatal mouse, scoop-shaped portion 10 is less than about 6 mm in width. In the case of a DOL28 neonatal mouse, scoop-shaped portion 10 is less than about 6-7 mm in width.

In addition to the appropriate sizing of scoop-shaped portion 10, it is important to ensure that scoop-shaped portion 10 does not contain any sharp corners or edges that could hurt or injure the animal. The edges of scoop-shaped portion 10 are smooth, so as to avoid injury to the animal and cause discomfort. The shape of scoop-shaped portion 10 is also selected to avoid significant discomfort in the animal, and generally contains round or curved edges.

The handle 12, shaft 9 and scoop-shaped portion 10 of the collection wand can all be manufactured from the same material. Alternatively, some or all of these components can be manufactured from different materials that are presently or later known in the art for collecting and handling oral samples. Suitable materials for scoop-shaped portion 10 include plastics, metals and alloys. The handle 12 and shaft 9 can also be manufactured from plastics, metals, alloys, wood, etc. In various embodiments the scoop is manufactured from a material that facilitates removal of a sample from the scoop surface, for example, polyurethane, PTFE, high density polyethylene (HDPE), Teflon, etc. Desirably, collection wand 4 is manufactured using material that permits sterilization.

### Oral Speculum

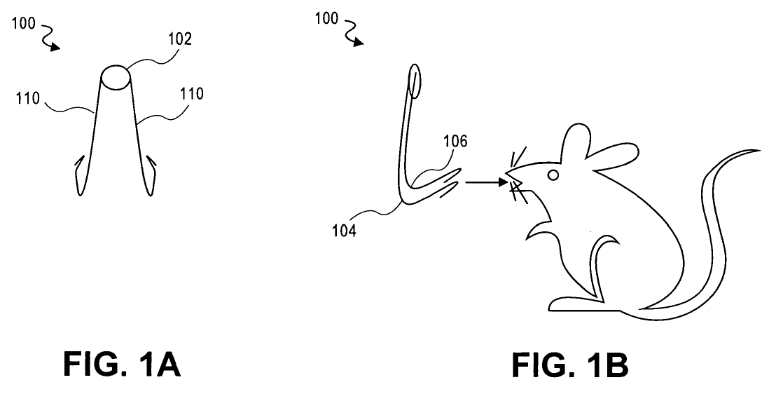

In accordance with one embodiment of the present invention, the collection assembly includes an oral speculum that is useful to maintain the animal's mouth open during sample collection. The oral speculum can be of the type presently or later known in the art for use with animals. In a specific embodiment the oral speculum is of the type described below and depicted in FIG. 1.

Referring to FIGS. 1A & B, oral speculum 100 comprises resilient biasing portion 102 having a first end comprising first speculum blade 104 and a second end comprising second speculum blade 106. Resilient biasing portion 102 urges first speculum blade 104 and second speculum blade 106 from a closed position to an open position. First speculum blade 104 and second speculum blade 106 are sized to fit within the mouth of an animal, wherein in said open position the distance between said first speculum blade and said second speculum blade is sufficient to maintain the mouth of said animal open. In accordance with the present invention, oral speculum 100 is configured to maintain the animal's mouth open without causing discomfort, or without causing significant discomfort, to the animal.

Biasing portion 102 also acts as handle for the operator. Arms 108 and 110 connect biasing portion 102 to first speculum blade 104 and second speculum blade 106, respectively.

In use, pressure applied to arms 108 and 110, for example by the operator during use, causes first speculum blade 104 and second speculum blade 106 from the open position towards the closed position. When first speculum blade 104 and second speculum blade 106 are in the closed position or in a position intermediate between the closed and open position, first speculum blade 104 and second speculum blade 106 can be inserted into the mouth of the animal to be sampled. Release of pressure from arms 108 and 110 allow first speculum blade 104 and second speculum blade 106 to move to the open position and, thereby, open the mouth of the animal to be sampled to permit oral sample collection.

In the example in which the animal from which a sample is to be obtained is a neonatal mouse, the distance between first speculum blade member 104 and second blade member 106 of oral speculum 100 in the open position is no more than about 3 mm. In the case of a DOL2 and DOL3 neonatal mouse, the distance is no more than about 3 mm. In the case of a DOL7 neonatal mouse, the distance is no more than about 5 mm. In the case of a DOL14 neonatal mouse, the distance is no more than about 5 mm. In the case of a DOL21 neonatal mouse, the distance is no more than about 6 mm. In the case of a DOL28 neonatal mouse, the distance is no more than about 6-7 mm.

Biasing portion 102 and arms 108 and 110 are sized to permit manipulation of first speculum blade 104 and second speculum blade 106 by an operator to maintain the animal's mouth open during sample collection. The shape and length of biasing portion 102 and arms 108 and 110 will vary according to the intended use, needs, preferences and/or choice of materials.

The size and shape of first speculum blade 104 and second speculum blade 106 of oral speculum 100 will also vary according to the intended use, needs, preferences and/or choice of materials. In one example, first speculum blade 104 and second speculum blade 106 of oral speculum 100 are substantially the same size. In addition to the appropriate sizing of first speculum blade 104 and second speculum blade 106 based on the animal to be sampled, it is important to ensure that neither speculum blade contain any sharp corners or edges that could hurt or injure the animal. The edges of the speculum blades are smooth, so as to avoid injury or discomfort in the animal. The shape of the speculum blades is also selected to avoid significant discomfort in the animal, and generally contains round or curved edges.

Oral speculum 100 is made from a pliable and firm material. Handle portion 102, first speculum blade 104 and second speculum blade 106 can all be manufactured from the same material. Alternatively, some or all of these components can be manufactured from different materials that are presently or later known in the art for collecting oral samples. Suitable materials for all or one of the components include plastics, metals and alloys. In a specific example, oral speculum 100 is made from a metal alloy wire. Desirably, oral speculum 100 is manufactured using material that permits sterilization.

### Collection Tube

In accordance with one embodiment of the present invention, the collection assembly includes a collection tube for storing the oral sample. In this embodiment, collection wand 4 is configured to fit within the collection tube.

Referring to FIGS. 2B & C, collection tube 14 includes a proximal end 30 having an opening 32 and a distal end 34. Optionally, handle 12 of collection wand 4 can be configured to sealing fit within opening 32 at the proximal end 30 of collection tube 14. Alternatively, handle 12 comprises a lid 16 adapted for removable attachment at the proximal end 30 of collection tube 14. In a specific example, lid 16 includes internal threads (not visible) for mating with external threads 38 if present at proximal end 30 of collection tube 14. Alternatively, a separate cap (not shown) can be used for sealing of collection tube 14.

In one example, collection tube 14 is a microcentrifuge tube, of the type that would be known to the skilled worker.

The collection tube 14 can be manufactured from any suitable material including a polymeric material, such as plastics commonly used in the art for similar sample material collection and/or performance of analyses as will be apparent from reading the present disclosure. Examples of suitable plastics include polyurethane, polystyrene, polyvinyl, polypropylene, polyurethane, etc. Desirably, the sample collection assembly, including collection wand 4 and collection tube 14, is amenable to sterilization during or after manufacture either as an assembled unit or as separate components.

### Collection Method

In accordance with another aspect of the present invention there is provided a method of obtaining an oral sample from an animal, such as a laboratory animal, for example, a neonatal laboratory animal.

The method generally comprises the steps of opening the mouth of the animal to be sampled, inserting scoop-shaped portion 10 of collection wand 4 into the animal's mouth, moving or rotating scoop-shaped portion 10 of collection wand 4 within the animal's mouth to obtain the oral sample and extracting scoop-shaped portion 10 of collection wand 4 together with the obtained sample from the animal's mouth. Such movement or rotation can additionally include the step of rubbing or scraping a surface within the mouth of the animal, such as, but not limited to, the inner cheek, tongue, roof of the mouth, sublingual area and gums.

In the example in which the collection assembly includes collection tube 14, collection wand 4 is removed from collection tube 14 and passed into the mouth of an animal. The handle 12 can be moved or rotated to facilitate collection of an oral sample into scoop-shaped portion 10. After removal from the animal, scoop-shaped portion 10 of collection wand 4 is passed through opening 32 of collection tube 14.

If handle 12 is configured to seal opening 30, the sample may be shipped to the lab sealed within collection tube 14. Alternatively, if handle 12 includes a threaded lid, lid 12 can be threaded onto threads 38 on the exterior of collection tube 14 to seal the sample within collection tube 14.

In a specific embodiment of the present invention, scoop-shaped portion 10 of collection wand 4 is used to collect an oral sample from the inner cheek (such as the cheek pouches/buccal cavity) of a neonatal mouse. In this example, scoop-shaped portion 10 is used to collect buccal mucosa containing buccal cells. It will be clear that in addition to, or alternatively, the oral sample may be obtained from other areas of the mouth of the animal, such as the tongue, gums, roof of the mouth, sublingual area, palate and/or the like.

In the specific example of a neonatal mouse, the oral sample may be collected by gently rubbing or scraping the cheek pouch of the animal.

One method of collecting an oral sample from an animal, comprises (a) maintaining the mouth of the animal open and (b) collecting the oral sample from the mouth of the animal using the collection assembly of the present invention. In this example, step (b) comprises inserting scoop-shaped portion 10 of collection wand 4 into the mouth of the animal, and collecting the oral sample from within the mouth of the animal. In a specific example, scooped-shaped portion 10 is used to collect buccal mucosa, containing buccal cells. As set out above, in addition to, or alternatively, the oral sample can be obtained from one or more other areas of the mouth of the animal, such as the tongue, gums, roof of the mouth, sublingual area, cheek, cheek pouch/buccal pouch, palate and the like. In one example, step (b) comprises collecting the oral sample from the cheek, cheek pouch/buccal pouch and/or tongue of the animal.

In one example, step (a) comprises inserting first speculum blade 104 and second blade 106 of oral speculum 100 into the mouth of the animal while in the closed position or a position intermediate between the open and closed position of oral 100 speculum and moving said first speculum blade and said second speculum blade to the open position thereby maintaining the mouth of the animal open. Alternatively, the mouth of the animal is maintained open without use of oral speculum 100.

A sample collection device according to the invention can be used for collecting oral samples that can be analyzed using, for example, PCR analysis, other methods of nucleic acid-based diagnostics, immunochemistry, biochemical analysis, microbial culture, mass spectrometry, and biosensor-based detection etc. Embodiments of the invention, however, are not limited to these examples. One of ordinary skill in the art will appreciate other types of analyses which may be performed on samples collected by the sample collection device disclosed herein.

In the case in which the method of the present application is used to isolate nucleic acids from the oral sample, the method further comprises the step of (c) isolating nucleic acid contained within the oral sample.

The term “nucleic acid”, as used herein, refers to a chain of nucleotides and includes deoxyribonucleic acid (DNA) or ribonucleic acid (RNA), typically found in chromosomes, chromatin, mitochondria, ribosomes, bacteria or viruses. In one example, the nucleic acid isolated by the method of the present invention is DNA or RNA. In a specific example, the isolated nucleic acid is DNA.

It will be clear that although the isolation of nucleic acids from an oral sample is exemplified, the present invention is suitable for the isolation and/or analysis of other compounds present within the oral sample. Non-limiting examples of such other compounds include peptides, proteins, hormones, chemicals and the like.

The present methods are also suited to generating cell lines from cells within the oral sample collected.

Methods of the invention are conveniently practiced by providing the collection assembly, including collection wand 4 and, optionally, one or both of oral speculum 100 and collection tube 14 in the form of a kit. Such a kit preferably contains appropriate instructions for use. Additionally, in the case in which the oral sample is to be used in a downstream PCR, the kit can also include primers and/or one a positive control useful in the detection of a target gene, DNA, RNA.

In one example, a kit comprises (a) collection wand 4 and (b) instructions for the use thereof. In another example, a kit comprises (a) collection wand 4, (b) oral speculum 100 and (c) instructions for the use thereof. In third example, a kit comprises (a) collection wand 4, (b) collection tube 14, (c) oral speculum 100 and (d) instructions for the use thereof.

To gain a better understanding of the invention described herein, the following examples are set forth. It should be understood that these examples are for illustrative purposes only. Therefore, they should not limit the scope of this invention in any way.

### EXAMPLE

This example describes the design and use of a mouse mouth collection assembly according to a specific embodiment of the present invention. This assembly can be used without causing discomfort, or without causing significant discomfort, to the mouse. Veterinarians and researchers look for the least harmful and least invasive method for procedures on an animal.

### Materials and Methods

### Animal Model

Gyk KO mice were obtained from W. J. Craigen at Baylor College of Medicine (2). They were bred and housed under an Animal Research Committee (ARC) approved protocol at the University of California, Los Angeles (UCLA). All experiments were performed according to a UCLA ARC approved protocol. DOLL neonatal mice were used for mouth cell sampling.

### Genotyping

Primers for genotyping were neo-F2 (5′-gcgcatcgccttctatcgcc-3′) and GykR (5′-gttcaagactccacacaccaacc-3′), which amplified the neomycin-Gyk junction fragment and primers for the normal allele, were GykF (5′-gatgccatgaatcgcgactgt-3′) and GykR (3). Sex-typing primers were developed to detect the X and Y chromosomes: primer #1 (5′-ccgctgccaaattctttgg-3′) and primer #2 (5′-tgaagcttttggctttgag-3′). A female produces 1 340 bp band whereas a male produces 2 bands: 301 and 340 bp.

### Mouth Cell Collection Process

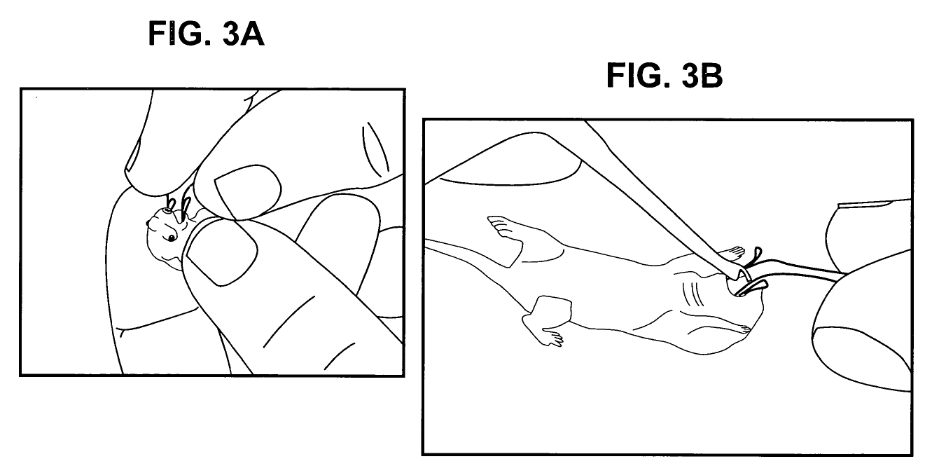

FIG. 1 depicts one example of oral speculum 100. FIG. 2 depicts an example of the collection assembly of the present invention including collection wand 4 and collection tube 14. FIG. 3 depicts a use of oral speculum 100 and collection assembly with a DOL1 mouse. To collect an oral sample, oral speculum 100 is inserted into the mouth of the neonatal mouse, and first speculum blade 104 and second speculum blade 106 are moved from a closed or nearly closed position to the open position, and thereby exert gentle pressure to open the mouth and maintain the mouth open during the sample collection procedure. In this example, the collection assembly comprises a 1.5 ml screwcap microcentrifuge tube with a 3 cm collection wand 4 fused to the lid (FIGS. 2B & 2C). The collection end of the sample collection wand has a spoon-shaped scoop and acts as a collection reservoir for collecting an oral sample; in this example buccal cells are scraped from the mouse cheek. Scraping the buccal mucosa on the inside of the mouse cheek can remove a large number of cells. The lid with the attached probe is sufficiently large to facilitate manipulation in the oral cavity of the neonatal mouse. For the collection procedure, a sample collection wand with integrated scoop was used to lightly rub 3-5 mm of the buccal mucosa in each neonatal mouse cheek pouch twice. The sample collection wand was removed from each mouse mouth concave side up and placed in the microcentrifuge tube containing 1 mL of normal (0.9%) NaCl. The sample collection assembly was used to seal the tube and was vortexed to suspend the cells in the saline.

### DNA Extraction

Each cell suspension was centrifuged at 14,000 rpm (15,800 g) for 10 sec and the supernatant discarded. 50 μL of lysis solution (25 mM NaOH, 0.2 mM EDTA) was added to each cell pellet and vortexed briefly. The resuspended pellet was heated at 98° C. for 20 min, vortexed briefly, and placed on ice. 50 μL of 40 mM Tris-Cl (pH 5.0) was added to neutralize the lysis solution and yielded a total volume of 100 μL.

### PCR

Two (2) μL of neutralized lysis extract was taken as an aliquot for PCR (total PCR volume 25 μL). Genotyping PCR for Gyk genotyping was performed according to a previously published protocol (3). Sex-typing PCR was performed as follows: 95° for 3 minutes; then 30 cycles of 94° for 30 seconds, 60° for 1 minute and 72° for 3 minutes; and finally an extension at 72° for 5 minutes.

### Results

For genotyping transgene and knockout mice, reduced pain and suffering for the mice, the ability to obtain DNA early in life, as well as efficiency and accuracy are critical. In many cases as in the murine model for human glycerol kinase deficiency (GKD), the glycerol kinase (Gyk) knockout mouse may be the only non-human mammalian model of GKD available. Gyk knockout mice require immediate genotyping, because they die at DOL 3-4. The methods of the present invention were successfully used to obtain sufficient DNA from buccal scrapings of mouse pup's as young as DOL1 to genotype and sex-type them for use in investigations before the knockout mice die (Table 1).

TABLE 1
Summary table: DNA obtained from mouse pups and pup weights.

| DNA | Average | Minimum | Maximum |
|---|---|---|---|
| concentrations (ng/ul) | 10 | 5 | 15 |
| Total DNA (ug) | 1.0 | 0.5 | 1.5 |
| In total volume 100 ul | | | |
| Pup Weight (g) | 1.30 ± 0.20 | 0.89 | 1.66 |

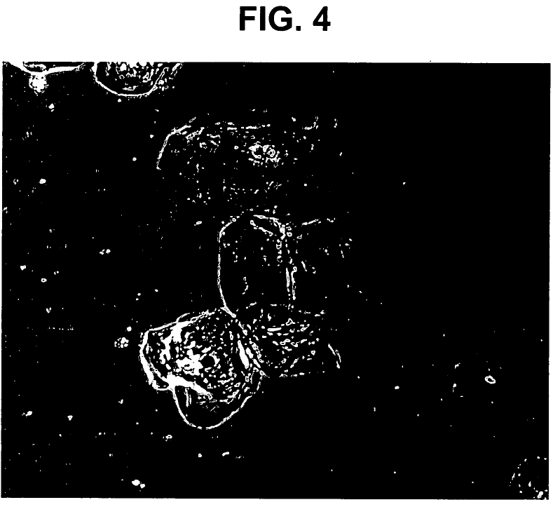

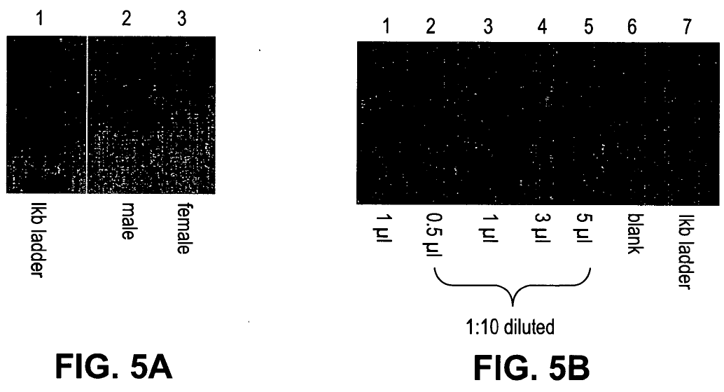

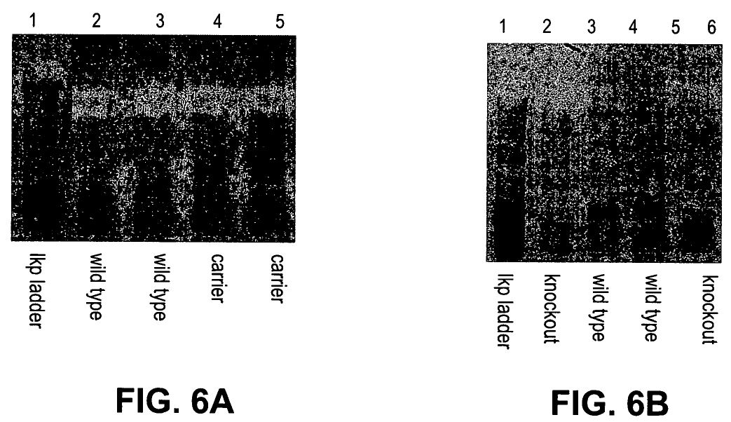

Enough DNA for up to 50 PCR reactions from each buccal sampling. FIG. 4 shows stained buccal cells obtained using the sample collection assembly. FIG. 5.A shows sextyping PCR results from amplified DNA obtained from male and female neonatal mouse pups. This DNA was diluted to various concentrations and successfully amplified as well (FIG. 5.B) showing signal from <1 μl of a 1:10 dilution of the 1001 PCR amplifications. These results show that at least 100 amplification reactions can be obtained from a single buccal collection in a newborn mouse pup. Genotyping PCR was performed to distinguish wild type, carrier and knockout DOLL neonatal mice (FIGS. 6A and 6B). These results were confirmed by trial biopsies (data not shown). These procedures were successfully replicated for 50 mouse pups.

The devices and methods of the present application permit retrieval of an oral sample from newborn mice (1.3 gram average weight). In a specific example, sample collection assembly is a sterile, disposable apparatus, configured to collect buccal cells from neonatal mice with no significant discomfort as measured by lack of significant contortions or back and forth body movements. In this specific example, use of the sample collection assembly yielded a high concentration of buccal cells for DNA extraction, and demonstrated by the fact that significant DNA can be extracted suitable for at least 50 PCR reactions.

The devices and methods of the present application are a minimally invasive, humane approach to cell collection.

### REFERENCES

1. Burger, M. F., E. Y. Song and J. W. Schumm. 2005. Buccal DNA samples for DNA typing: new collection and processing methods. Biotechniques 39:257-261.
2. Huq, A. H., R. S. Lovell, C. N. Ou, A. L. Beaudet and W. J. Craigen. 1997. X-linked glycerol kinase deficiency in the mouse leads to growth retardation, altered fat metabolism, autonomous glucocorticoid secretion and neonatal death. Hum Mol Genet 6:1803-1809.
3. Kuwada, N., K. Nagano, N. MacLennan, J. Havens, M. Kumar, K. M. Dipple and E. R. McCabe. 2005. Gene therapy for murine glycerol kinase deficiency: importance of murine ortholog. Biochem Biophys Res Commun 335:247-255.
4. Meldgaard, M., P. J. Bollen and B. Finsen. 2004. Non-invasive method for sampling and extraction of mouse DNA for PCR. Lab Anim 38:413-417.
5. Michalczyk, A., G. Varigos, L. Smith and M. L. Ackland. 2004. Fresh and cultured buccal cells as a source of mRNA and protein for molecular analysis. Biotechniques 37:262-264, 266-269.
6. Mulot, C., I. Stucker, J. Clavel, P. Beaune and M. A. Loriot. 2005. Collection of human genomic DNA from buccal cells for genetics studies: comparison between cytobrush, mouthwash, and treated card. J Biomed Biotechnol 2005:291-296.
7. Spira, A., J. Beane, F. Schembri, G. Liu, C. Ding, S. Gilman, X. Yang, C. Cantor and J. S. Brody. 2004. Noninvasive method for obtaining RNA from buccal mucosa epithelial cells for gene expression profiling. Biotechniques 36:484-487.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

The embodiments of the invention in which an exclusive property or privilege is claimed are defined as follows:

1. A sample collection assembly, comprising:
a collection wand having an operation end and a collection end, said collection end having a scoop-shaped portion configured for collecting an oral sample from within the mouth of an animal wherein said scoop-shaped portion is sized and shaped to fit within the mouth of said animal without causing significant discomfort to said animal.

2. The assembly of claim 1, wherein said oral sample is collected from an inner cheek, cheek pouch/buccal pouch, palate and/or tongue of the mouth cavity of said animal.

3. The assembly of any one of claims 1-2, wherein said oral sample is sputum, saliva, microorganisms, cells, buccal mucosa or any combination thereof.

4. The assembly of any one of claim 1-3, wherein said animal is a neonatal animal.

5. The assembly of claim 4, wherein said animal is a neonatal mouse, a neonatal rat, a neonatal hamster, or a neonatal rabbit.

6. The assembly of claim 5, wherein said animal is a neonatal mouse and said scoop-shaped portion is less than about 2 mm in width, less than about 3 mm in width, less than about 5 mm in width, less than about 6 mm in width or less than about 7 mm in width.

7. The assembly of any one of claims 1-6, wherein said operation end comprises a handle configured to permit a user to manipulate the collection wand in the mouth of the animal.

8. The assembly of any one of claims 1-7, which additionally comprises a collection tube.

9. The assembly of claim 8, wherein said operation end of said collection wand includes a lid portion adapted for removable attachment to said collection tube such that when said lid is attached to said collection tube the collection end of said wand is within said collection tube.

10. The assembly of claim 8, wherein said collection tube includes a removable cap.

11. The assembly according to any one of claims 7-10, wherein said collection tube is a microcentrifuge tube.

12. The assembly according to any one of claims 1-11, wherein said collection wand is about 3 cm in length.

13. The assembly according to any one of claims 1-12, wherein said collection wand is made from a plastic, a metal, an alloy, or any combination thereof.

14. An oral speculum, comprising:
a resilient handle portion having a first end comprising a first speculum blade and a second end comprising a second speculum blade, said resilient handle urges said first speculum blade and said second speculum blade from a closed position to an open position, said first speculum blade and said second speculum blade are sized to fit within the mouth of an animal,
wherein in said open position the distance between said first speculum blade and said second speculum blade is sufficient to gently maintain the mouth of said animal open.

15. The speculum of claim 14, wherein said animal is a neonatal animal.

16. The speculum of claim 15, wherein said animal is a neonatal mouse, a neonatal rat, a neonatal hamster or a neonatal rabbit.

17. The speculum of claim 16, wherein said animal is a neonatal mouse and the distance between said first speculum blade member and said speculum second blade member in the open position is no more than about 3 mm.

18. The speculum according to any one of claims 14-17, wherein the speculum is made from a pliable and firm material.

19. The speculum according to any one of claims 14-18, wherein the speculum is made from a plastic, a metal, a metal alloy, or any combination thereof.

20. The speculum according to any one of claims 14-19, wherein the speculum is made from a metal alloy wire.

21. The speculum according to any one of claims 14-20, wherein said first speculum blade and said second speculum blade are substantially the same size.

22. The speculum according to any one of claims 14-21, wherein said handle portion is sized to permit manipulation of said first speculum blade and said second speculum blade by a user in the mouth of the animal.

23. The assembly according to any one of claims 1-13, additionally comprising an oral speculum according to any one of claims 14-22.

24. A method of collecting an oral sample from an animal, comprising:
(a) maintaining the mouth of the animal open;
(b) collecting the oral sample from the mouth of the animal using a collection assembly according to any one of claims 1-13 or 23.

25. The method of claim 24, wherein step (b) comprises inserting the scoop-shaped portion of said collection wand into the mouth of the animal, and collecting the oral sample from within the mouth of the animal.

26. The method of claim 25, wherein step (b) comprises collecting the oral sample from the cheek and/or tongue of the animal.

27. The method of any one of claims 24-26, wherein step (a) comprises inserting the first and second blades of the speculum according to any one of claims 14-22 into the mouth of the animal in the closed position or in a position intermediate between the closed position and the open position and moving said first speculum blade and said second speculum blade to the open position thereby maintaining the mouth of the animal open.

28. The method according to any one of claims 24-27, additionally comprising the step of (c) isolating nucleic acid contained within the oral sample.

29. The method according to claim 28, wherein said nucleic acid is DNA or RNA.

30. The method of any one of claims 24-29 additionally comprising the step of subjecting nucleic acid within the oral sample to genotyping.

31. A kit comprising.
(a) a collection assembly according to any one of claims 1-13 or 23; and
(b) instructions for the use thereof.

32. The kit according to claim 31, further comprising the oral speculum according to any one of claims 14-22.
